(19) United States
(12) Patent Application Publication
(10) Pub. No.: US 2008/0281424 A1
(43) Pub. Date: Nov. 13, 2008

(54) Orthopaedic Implants and Prostheses

(76) Inventors: John Parry, Worcs (GB); David Marks, Birmingham (GB)

Correspondence Address: Beusse Wolter Sanks Mora & Maire, 390 N. ORANGE AVENUE, SUITE 2500, ORLANDO, FL, 32801, US

(21) Appl. No.: 11/946,975

(22) Filed: Nov. 29, 2007

Related U.S. Application Data
(60) Provisional application No. 60/867,627, filed on Nov. 29, 2006.

(30) Foreign Application Priority Data
Nov. 29, 2006 (GB) 0623801.8

(51) Int. Cl. A61F 2/44; A61B 17/04; A61B 17/08

(52) U.S. Cl. 623/17.16; 623/17.11; 606/324; 606/151

(57) ABSTRACT

The invention relates to an implant for repairing a damaged body structure that comprises or is associated with bone parts. In one aspect a spinal implant includes an inferior member having an inferior end surface for engaging a superior face of an inferior vertebral body and a longitudinal portion; a superior member having a superior end surface for engaging an opposing inferior surface of a second vertebral body, and a portion adapted to cooperate with the longitudinal portion of the inferior member such that the superior member is moveable relative to the inferior member by sliding in the longitudinal direction; and fixating means for securing the superior member to the inferior member. Also described are instruments and methods used in the repair of such damaged body structures.

### RELATED APPLICATIONS

This applications claims priority to U.S. Ser. No. 60/867,627 filed Nov. 29, 2006 and UK application number 0623801.8 filed Nov. 29, 2006, which are incorporated herein in their entirety.

### TECHNICAL FIELD

The present invention relates to orthopaedic implants and/or prostheses and instrumentation for their implantation. The invention is applicable to bone structures, particularly the cervical, thoracic and lumbar spine

### BACKGROUND

Bones and related structural body parts, for example spine and/or vertebral bodies and/or intervertebral discs, may become crushed or damaged as a result of trauma/injury, or damaged by disease (e.g. by tumour, auto-immune disease), or damaged as a result of degeneration through an aging process. In many such cases the structure can be repaired by replacing the damaged parts (e.g. vertebra and/or discs) with a prosthesis or implant. A method of repair is to remove the damaged part(s) (e.g. vertebra and/or partial vertebra and/or disc and/or partial disc) and replace it with the implant such that the implant is free standing or fastened in position between adjacent undamaged parts (e.g. adjacent vertebral bodies).

There are situations where the patient has previously had an implant or arthrodesis implanted but for reasons such as implant or arthrodesis failure, or incorrect positioning of the implant or arthrodesis, there is a need to remove the implant or arthrodesis and replace it with a new implant in order to stabilise the bone structure or reduce the level of pain and/or increase mobility.

Associated with this method of repair, is fusion of the bone structure where the implant is placed. Typically an implant may consist of a central space surrounded by a continuous wall that is open at each end (e.g. superior and inferior). This form of implant is thought to allow bone to develop within the central space, developing from each extremity of the implant towards the centre. Typically an implant shall be secured directly to a bone structure by mechanical or biological means.

Many current implants and prostheses are hollow to allow bone to grow within the hollow space. One problem identified by the inventors, when replacing large structural sections, is that the relationship of length (or height) to cross sectional area of the central space is large. The larger this relationship, the more a problem arises in providing an adequate blood supply and nutrients to allow fusion and or bone growth into the hollow centre to take place, either in a timely manner, or at all. One conventional solution to this problem is to make the central space have as large a cross section as possible. However, this is limited by the wall thickness and the material used for the implant, which determine its mechanical strength.

For this reason, orthopaedic surgeons will often pack the space within the implant with an injectable or mouldable bone growth material or with fragments of bone taken from other parts of the patients body i.e. autograft or bone from biocompatible sources, for example allograft or synthetic bone. The inventors have realized that even then there may not be complete fusion of the implant into the bone structure.

Another problem arises because many current implants and prostheses are formed of metal in order to have the required structural properties (i.e. strength). A problem with metals is that they are opaque to X-rays, and so obscure parts of the spine for example in assessing the rate of fusion when using X-radiography.

Another problem of metal is that the Modulus of Elasticity is much higher than the bone structure to which it is secured. This creates a relatively higher stiffness resulting in stresses being transferred to adjacent bone structures, for example an adjacent vertebra and potential stress fractures through stress shielding and bone graft resorbtion.

Although the following discussion focuses on spinal implants or prostheses, it will be appreciated that many of the principles may equally be applied to other bone structures within the human or animal body.

It is known to provide an implant as a replacement for vertebral body or VBR device. Examples such implants are described in U.S. Pat. No. 6,524,341 and U.S. Pat. No. 6,176,881). These, and similar implants and prostheses are able to be telescoped to replace a vertebral body/disc in the spine. However, the inventors have recognized that such devices are difficult at best to be positioned, telescoped and secured at the desired height. The inventors have endeavoured to develop a VBR device that is easier and safer to use in the vital spine area, and which leads to a more beneficial surgical outcome.

Another problem arises, particularly with spinal implants and prostheses, because the size of the space into which the implant is to be inserted varies from patient to patient and also depends on its position in the bone structure, e.g. the spinal column. One conventional solution to this problem is to have multiple shapes and sizes of implant. However, this results in intra-operative complexity and a large, hence expensive, range of stock. Another conventional solution to this problem is to have an implant that is adjustable, e.g. the height, width or angle is adjustable. This adjustable height may be achieved through, for example, mechanical, hydraulic or pneumatic means. There are various designs with adjustable height on the market or described in literature, such as the use of dampers e.g. springs (Intervert Locking Device, described in U.S. Pat. No. 5,360,430), or a compressible core (Trieu, Compressible Corpectomy Device, described in US 2005/096744) or the use of liquids (Barber, Vertebral Body Prosthesis, described in U.S. Pat. No. 5,236,460), or the use of stackable building blocks (DePuy, Stackable Cage described in U.S. Pat. No. 6,159,211), or the use of adjustment by a screw principle (Berry, US 2004/0186569). However, the inventors have realized a problem with each of these adjustable height devices exists as a result of the complexity of the procedure required to insert and then adjust the height of the implant.

Another problem, particularly with the spine, is that the damaged or degenerate structure (vertebral body) collapses and the space into which the implant is to be inserted needs to be expanded prior to insertion. One conventional solution to this problem is to open up the space using a suitable distracting instrument and then to insert a number of implants in layers so as to fill the space before the instrument is removed (US 2005/187625). However, the inventors have determined that this is a relatively complex multi-step procedure requiring the surgeon to fit multiple implants into one space, and then ensuring each layer is adequately connected together mechanically. Another conventional solution for repairing a damaged spine is to provide an expandable implant, which is inserted into the inter-vertebral space in a retracted condition, and is then expanded in the longitudinal direction until it engages the lateral faces of the adjacent vertebral bodies. This expansion opens up the inter-vertebral space, restoring the spine to its anatomical state, and ensures that the implant engages the adjacent vertebral body in a secure and rigid manner. One such expandable implant has a cylindrical form with two portions linked to each other by a screw thread. After insertion in the retracted condition, one portion is rotated relative to the other such that the screw connection causes the two portions to move axially and expand the implant (see Berry, US 2004/0186569).

One problem with these known expandable vertebral implants or prostheses identified by the inventors is that they require a two-stage operation. Firstly, the prosthesis must be inserted into an inter-vertebral cavity using one instrument. A second stage involves expanding the implants to the required height using a second instrument. Using two instruments increases the complexity of the procedure, increases the operative time, increases the patient risk and includes the risk of instruments clashing with each other within the small operative opening.

Another problem with such an implant based on a screw principle arises, as realized by the inventors, because it is necessary to rotate one portion to expand the implant after it has been inserted. This rotation can be difficult to achieve in such confined spaces. A further problem with such an implant is that it is more difficult to control the force exerted on an adjacent vertebral body during expansion because, for example the Clinician has no direct feel of the resistance force of the spine as the device is being expanded.

A further problem with these prostheses, as realized by the inventors, arises from their cylindrical shape e.g. they can twist inside each other which have poor inherent resistance to torsion. It is important for the implant to be locked in position between the adjacent vertebral bodies and resist relative movement even under torsion when a twisting movement is applied to the spine.

Another problem of an implant made from metal is that the Modulus of Elasticity is much higher than the vertebral body to which it is secured. This creates a relatively higher stiffness resulting in stresses being transferred to an adjacent vertebral body and potential stress fractures through stress shielding and bone graft resorbtion.

Another problem realized by the inventors is that the implant generally is not sufficiently secured to the spine and does not provide sufficient stability of the spine when used by itself alone. To achieve this necessary stability, the implant requires a second system such as a plating or rod based system that is attached to the spine and the implant. This additional system is not integrated, it is an additional cost, it may increase the operative time and risk to the patient.

Another problem is that implants or prostheses are generally manufactured from materials that are structurally acceptable but remain in the body for an indefinite period. Such metal implants designed for fusion have a Young's modulus greater than natural bone that may result in mechanical stress shielding in adjacent levels, leading to high stresses, deformation and/or fractures of the adjacent vertebral body.

### SUMMARY

Several embodiments of the present invention provide implants or prostheses, including vertebral prostheses, which alleviate the aforementioned problems.

According to a first aspect of the present invention there is provided an implant for repairing a damaged body structure comprising or associated with bone parts, wherein the implant is constructed from a bio-resorbable material and comprises one or more inner open porous structures to facilitate bone growth therein, and a higher density peripheral structure.

It will be understood that an open porous structure is one where the voids or pores are generally interconnected with one another (e.g. like a sponge) such that bone material can integrate e.g. can grow into and through the pores. A bio-resorbable material is one that, once implanted into the body is resorbed over a period of time by biochemical action of body fluids, so that eventually the entire implant is either dissolved or changed/replaced by natural body materials (e.g. bone or tissue). A higher density structure is meant as one that has less voids or pores than a lower density structure. A higher density structure will have greater strength than a lower density structure. Hence it is an advantage that the peripheral structure of the implant provides the required mechanical strength, while the inner, porous structure enhances the growth of bone material.

It is an advantage that the construction of the implant from a bio-resorbable material means that in time bone will grow into the implant and completely fuse the implant to neighbouring bone parts.

In a specific embodiment, the bio-resorbable material is an osteo-conductive material that encourages inward bone growth from the associated bone parts. In a more specific embodiment, the material is also osteo-inductive wherein bone can grow spontaneously within the material by way of a chemical reaction within the body. Even more specifically, the material is both osteo-conductive and osteo-inductive.

The inner structures and the peripheral structure may be integral. The implant may have a single structural component. The inner structures and the peripheral structure may be connected to each other. Alternatively, the inner structures and the peripheral structure may be separate from each other.

The implant may comprise more than one inner porous structure. Each inner porous structure may have a different density.

In a specific embodiment, the bio-resorbable material is an osteo-conductive material that encourages inward bone growth from the associated bone parts. In a more specific embodiment, the material is also osteo-inductive.

In one embodiment, the implant is a spinal implant for repairing a damaged disc. In a specific embodiment, the implant is configured to be inserted into an inter-vertebral space following a discectomy.

The implant may comprise one or more holes, one or more partial holes, grooves or slots for one or more fasteners to secure the implant to an adjacent vertebral body. Examples of fasteners include screws, pins, staples, darts, bollards or other suitable fixings. Anti-backout means may be provided to lock the fasteners in position so as to prevent back-out. The anti-backout means may be a primary means integral with the fastener or implant/prosthesis. Alternatively the anti-backout means may comprise a secondary device securable to the implant.

Further holes or openings may be provided for encouraging inward bone growth. In one embodiment, the implant has a generally square or rectangular cross-section. The implant may have walls surrounding a hollow central space. Alternatively, the implant may have a central space that is filled with a biological material such as autograft, allograft and/or synthetic bone material.

In another embodiment, the implant is a prosthesis for replacement of a partial or one or more entire vertebral bodies.

According to one embodiment invention, there is provided an implant comprising: a first inferior member having an inferior end surface for engaging a superior face of an inferior vertebral body and a longitudinal portion; a second superior member having a superior end surface for engaging an opposing inferior surface of a second vertebral body, and a portion adapted to cooperate with the longitudinal portion of the inferior member such that the superior member is moveable relative to the inferior member by sliding in the longitudinal direction; and fixating structure for securing the superior member to the inferior member.

In a specific embodiment, either the longitudinal portion of the inferior member or the cooperating portion of the superior member is an open-ended portion having a bore that receives the cooperating portion of the other member, so as to provide a close sliding fit.

In certain embodiments, the invention is especially configured for implantation via a lateral surgical approach or a midline (anterior) surgical approach.

The fixating structure between the inferior and superior members may be configured to allow adjustment of the height (or length) of the implant by securing the superior member to the inferior member at positions between a maximum and a minimum height (or length). This has the advantage that one size of implant may be usable in place of one or more complete or partial vertebral bodies in a wide range of spine sizes and positions.

Each member of the aforementioned implant is typically fixed directly to the adjacent superior and inferior vertebral bodies. In certain embodiments, the implant is reversible so as to be capable of placement in either an anterior orientation or a posterior orientation. In the anterior orientation the implant may provide a neutral or lordotic angle between the superior and inferior vertebral bodies, and, when reversed into the posterior orientation, provide a neutral or kyphotic angle.

In an alternative configuration, the superior and inferior members may have a symmetry of shape and angle of their respective end surfaces such that the implant can provide a blend of kyphotic and lordotic angles. For example, the implant may provide a lordotic angle on the superior end surface and a kyphotic angle on the inferior end surface. This results in reduced intra-operative complexity and reduces the range of inventory (i.e. the stock of different types/sizes of implant required to cater for different patients/injuries).

In an alternative configuration, the superior and inferior members provide a normal or neutral angle.

In a preferred embodiment, the first and second members are formed of a radio-translucent material, typically a polymer such as polyether-etherketone (PEEK). It is an advantage that parts of the spine are not obscured when X-rays are taken—e.g. to monitor fusion rates.

Embodiments may be formed of a polymer, polymer composite, bio-resorbable or other biomaterials. Preferably, the material exhibits the characteristics of Young's Modulus close to that of bone. Other preferred material properties include radiotranslucency and a mechanical strength adequate to stabilise the spine. The material may be a synthetic bone substitute, bioglass (such as phosphate glass) or ceramic material that is resorbable and/or osteoinductive and/or osteostimulative and/or osteoconductive.

The cross-sections of the inferior and superior members are preferably shaped to resist rotation relative to each other and relative to the adjacent bone structures. It is an advantage that the cross section provides high resistance to torsion.

The fixating of the inferior to superior members may comprise a clip or spring or threaded bolt, or pin(s) or staple(s) or other fasteners of similar shape and function. The implant may be configured for insertion of the fixating means into a hole or holes in the wall of the longitudinal portion of the inferior member so as to engage into a hole or holes formed in the longitudinal portion of the superior member. As an alternative to holes, there may be partial holes, grooves or channels formed in the cooperating surfaces of the longitudinal portions of the inferior and superior members for receiving the fixating means.

In one embodiment, the cross-section of the longitudinal portion of the superior member has a pair of substantially parallel flat faces, each face including a plurality of preformed holes or grooves extending laterally across the face, the preformed grooves being sized to receive an arm of the threaded bar or pin or staple. The holes or grooves or channel may be reinforced with a composite material or sleeve or similar. The superior and/or inferior members may be provided with an array of holes or grooves or channels to provide a range of insertion locations defined by alignment of one or more holes/grooves in the superior member with one or more holes/grooves in the inferior member. The pitch of the holes, or the increments at which the implant can be locked rigid, is small enough to support the surgical technique. The holes or grooves or channels and threaded bar or pin or staple may be of any suitable cross section, for example circular. The hole or holes in the wall of the longitudinal portion of the inferior member may be preformed, or formed as part of the insertion procedure.

It is an advantage that the height of the implant can be adjusted preferably in a parallel motion before the pin or staple is inserted through one member, and once in position at the required height, the pin with or without threaded portion or staple can be inserted to engage in a hole or groove in the other member. In an exemplary embodiment, the holes or groove or channel are spaced at intervals of up to 3.5 mm. In a preferred embodiment, the intervals are between approximately 1.5 mm and 3.5 mm, preferably about 2.5 mm, allowing adjustment of the height between approximately 1.5 mm and 3.5 mm steps. It will be appreciated that smaller or larger adjustment steps may be achieved by use of smaller or larger holes or grooves or channels, or by providing a plurality of preformed holes in the first member allowing a choice of locations through which the pin(s) or staple(s) can be inserted.

In a further embodiment, the inferior and superior members are secured to the corresponding vertebral body with screws, darts or bollards or fixing of similar function through pre-shaped holes in the members. This provides an advantage of immediate stability of the spine in resisting forces applied upon it.

In a further embodiment anchoring means are provided for anchoring the implant to an anterior or lateral plate or rod system. Preferably the plate or rod system is integrated with the implant. The anchoring means may comprise an opening in the first and/or the second member for receiving an end of a poly-axial coupling assembly. The poly-axial coupling assembly may include a clamping arrangement for securing the coupling to a longitudinal rod or bar forming part of the anterior plate, rod or other supplementary fixation system. It is an advantage that the use of the poly-axial coupling allows the implant to be anchored to the plate or rod system without requiring precise alignment. The invention also allows the use of posterior fixation as a means of additional spinal stability should the Clinician so desire. An alternative anchoring means may comprise of a hinged joint between the members and plate.

In another embodiment, means are provided for an expansion tool to engage the first and second members so as to move the members to increase or decrease the height of the implant prior to fixation. The expansion tool may engage the first and/or second member by insertion of one or more attachments of the tool into openings provided in the respective member. The expansion tool may be an instrument in accordance with the fourth aspect of the invention below.

According to a further aspect of the present invention, there is provided an instrument for inserting and expanding a vertebral implant, the instrument comprising: superior and inferior engagement arms for engaging respective superior and inferior members of said vertebral implant; input means distally of said engagement arms for operation by a practitioner to actuate said instrument; and means responsive to actuation for moving said arms apart to expand said implant while maintaining said arms in parallel alignment with one another.

Preferably, each arm comprises one or more attachments for engaging corresponding locations in said respective superior and inferior members of said implant. The attachments may comprise one or more fingers for insertion into corresponding holes in the members. In specific embodiments, the fingers possess a circular cross-section. However, other suitable cross-section shapes include elliptical or orthogonal cross-sections, or may comprise a combination.

In another embodiment, the attachments comprise clamping means for clamping the arms to the members.

The input means may comprise a pair of handle members that can be moved towards one another by squeezing in a practitioner's hand to actuate the instrument. A biasing means may be provided for providing a resistance to the squeezing together of the handle members so as to correlate to the expansion of the instrument.

Embodiments may further comprise a locking means for locking the instrument in an expanded position. The expanded position may be any position to which the instrument has been moved from a fully retracted position.

Embodiments may further comprise an aligned support member for supporting a fastener for fixating the vertebral implant in an expanded condition. The aligned support member may also include a guide means for locating a fixing tool (e.g. a screwdriver or the like).

It is an advantage that, in use, a surgeon can insert the implant into the spine and expand it to the required height in one operation using one instrument (for example a distracting tool). It is a further advantage that the surgeon can feel or gain a sense of the force resisting the expansion of the implant, and use this to gauge an appropriate amount of expansion. For example, the expansion tool with superior and inferior members of the implant attached, may be inserted into the spine in its collapsed position. Once each member is sitting in the correct anatomical position within the vertebral body, the distraction tool is activated such that the attachments are moved apart in a parallel motion until the required anatomical height is achieved. At this point the instrument can be locked to hold the implant in the expanded position, and fasteners inserted to fixate the implant by use of the aligned fastener support member. The instrument may feedback the value of the load resisting expansion through a display of force indicator. Once the implant has been fixated in position the instrument can be removed by simply pulling it away from the implant so that the fingers slide out of the holes in the superior and inferior members of the implant, or otherwise disengaging the instrument from the implant.

It is an advantage that the distraction tool can be used as a dual insertion and distraction instrument. It is also an advantage that use of the distraction tool is simple to operate and provides the practitioner with direct tactile feedback on assembly of both members, force or mechanical advantage to distract, relative position of holes or grooves in each member, and gauging the size of the members to use. A feature of the distraction tool is that the aligned support member can be used to indicate when the superior and inferior members are aligned correctly to accept the fixating fasteners(s).

Once each member has been distracted into its required anatomical position, the distraction tool provides an additional advantage of guiding the fixing tool e.g. screwdriver into the drive of the fastener e.g. pin, screw thread or staple.

According to a further aspect of an invention embodiment, there is provided a vertebral implant configured for securing a curtain at a plurality of positions on the implant so as to form an enclosed space between an upper vertebral body and a lower vertebral body, in which bone graft material may be contained. Bone graft may be allograft, autograft or synthetic. It may be in a granular form or semi or fully shaped, e.g. a rib strut.

The means for securing the curtain may comprise eyelets in the curtain for location onto lugs provided on the implant. The lugs are inserted onto the same apertures used by the distractor to expand the prosthesis. Alternatively, the curtain may have an open braid, web-like or netting structure and may be secured to the implant by locally deforming the open structure of the curtain to fit over an anchor point or lug.

The graft-retaining curtain is preferably formed of a biocompatible material with a structure to allow vascularisation and bone growth. The advantage of this is that the graft-retaining curtain retains the bone graft in position on the anterior face if laterally implanted for a trauma or tumour reason, or on its lateral sides if implanted anteriorly for a two level revision. Fusion is therefore promoted on the anterior faces of the vertebral body, being the faces that carry the highest spinal loading and that are most vascular, which creates the desired environment for fusion to take place. Suitable materials for the graft retainer are polyester, collagen (matrix/solid sheet), PEEK fibres or other low fractional polarity material. It is preferred that the retaining curtain has an open braid structure, small enough to retain fine and coarse bone graft or bone substitute but large enough to allow a blood supply with nutrients to flow in.

The graft-retaining curtain may contain an elastomeric section to facilitate attachment to the implant and to provide tensioning of the curtain or allowing expansion. In a specific aspect, the bone graft retaining curtain is radiolucent.

In a more specific embodiment the graft-retaining curtain is configured such that it can be securely attached to both members of the implant and then expanded/stretched with the device as it is expanded.

It is an advantage that, by forming an enclosure filled with bone graft material, the material is prevented from falling out. The bone graft material will promote bone growth, and in time will fuse to the vertebral body, thereby increasing the strength of the spine.

Bone graft material additionally may be inserted within the central portion of the implant.

It is another feature of this design that the implant can be explanted should there be a clinical need e.g. with inflammation due to infection.

### BRIEF DESCRIPTION OF THE DRAWINGS

The foregoing features of the invention will be more readily understood by reference to the following detailed description, taken with reference to the accompanying drawings.

FIG. 1 is an isometric view of a vertebral implant;

FIG. 2 is an elevation of a cross-section through the implant of FIG. 1;

FIG. 3 is a lateral view of the vertebral implant of FIG. 1;

FIG. 4 is an isometric view of a vertebral implant positioned within vertebral bodies;

FIG. 5 is an isometric view of a vertebral implant including a graft retaining curtain;

FIG. 6a-c is an isometric view of a vertebral implant having partial holes grooves or channels and coupled to an anterior plate system, this is an alternative design to the holes in the wall as shown in FIGS. 1-4.

FIG. 7 a-d show several different views of an embodiment especially adapted for an anterior midline surgical approach. FIG. 7a represents a planar top view of the embodiment, FIG. 7b represents an anterior side view of the embodiment, FIG. 7c represents a lateral side view of the embodiment, and FIG. 7d represents a perspective view of the embodiment.

FIG. 8 shows an embodiment representing the angulation of the superior member, which may also apply to the inferior member.

FIG. 9. shows an embodiment representing a configuration of the superior member to provide for an angulation similar to that of FIG. 8.

FIG. 10 depicts an instrument for inserting and expanding an implant or prosthesis;

FIG. 11 is a detailed view showing part of the instrument of FIG. 13 and an expendable implant in position between vertebral bodies; and

FIG. 12 depicts the instrument of FIG. 11 in association with a fastener inserting device.

FIG. 13 depicts another fastener inserting device.

### DESCRIPTION OF ILLUSTRATIVE EMBODIMENTS

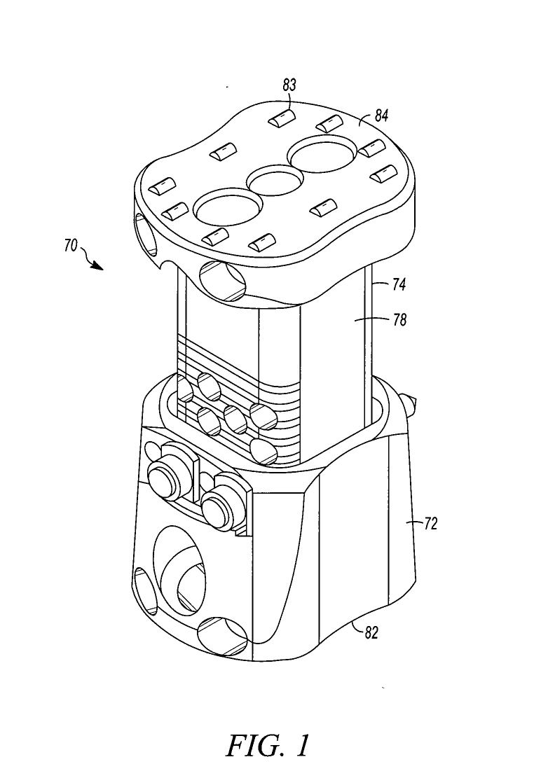

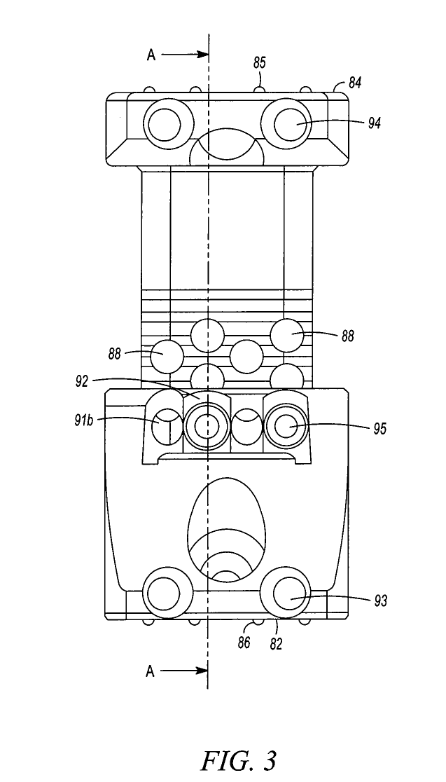

Referring to FIGS. 1 to 3, an inter-vertebral prosthesis 70 has a first member 72 and a second member 74. The first member 72 is of a tubular construction having a bore 76. The second member 74 has a longitudinal portion 78 having a cross-section that forms a close sliding fit in the bore 76 of the first member. The second member 74 can slide in the longitudinal direction (up and down as shown) relative to the first member 72. In one embodiment, the first and second members are potentially formed of PEEK.

The first member 72 will be referred to hereafter as the inferior member and the second member 74 as the superior member, although it will be appreciated that the prosthesis 70 may operate equally well if turned upside down. The inferior member 72 has an end surface 82 for engaging an opposing superior face of a first vertebral body (not shown). The superior member 74 has an end surface 84 for engaging an opposing inferior face of a second vertebral body (not shown). Each of the end surfaces 82, 84 is provided with raised teeth, 83, 85, 86, which may be elongated teeth, or ridges for gripping the bone of the vertebral body. Alternatively the end surfaces 82, 84 may be provided with grooves or raised protrusions, such as pyramids to provide a gripping action.

The cross-sections of the bore 76 and the longitudinal portion 78 of the superior member 74 are non-circular, to resist relative rotation between the superior and inferior members. The embodiment shown has a pair of opposing faces on the bore 76 and corresponding faces 89a, 89b on the flanks of the longitudinal portion 78.

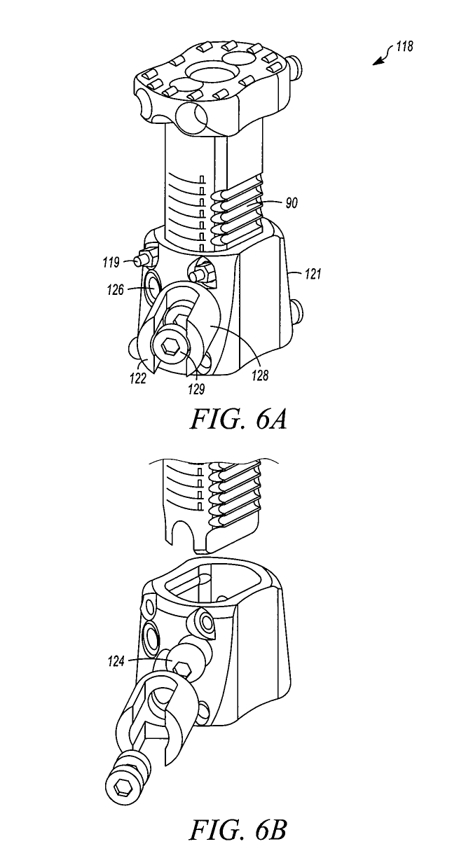

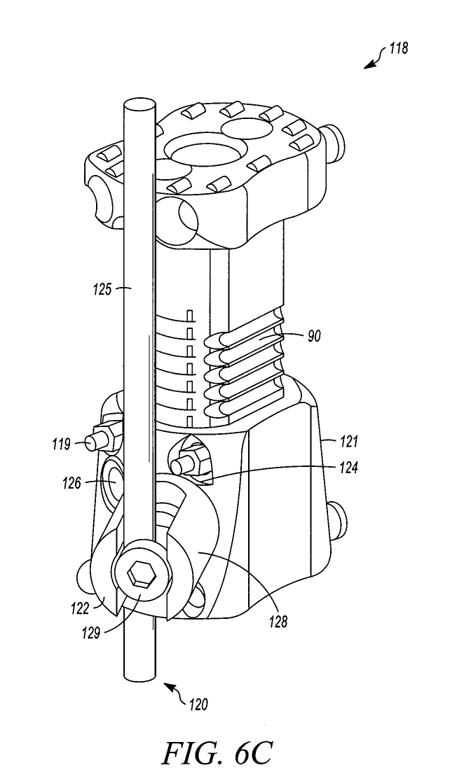

A matrix of horizontal holes 88 is provided in one or both of the faces 89a, 89b of the superior member 74. The holes are spaced at intervals of e.g. 2.5 mm in the longitudinal direction. Alternatively, the faces 89a, 89b may each be provided with a series of horizontal grooves (as shown in FIG. 6) 90, spaced at intervals of e.g. 2.5 mm. It will be appreciated that any suitable or desirable spacing of the holes 88 or grooves 90 may be provided to allow adjustment in desired increments. A preferred spacing would be in the range 1.0 to 4.0 mm.

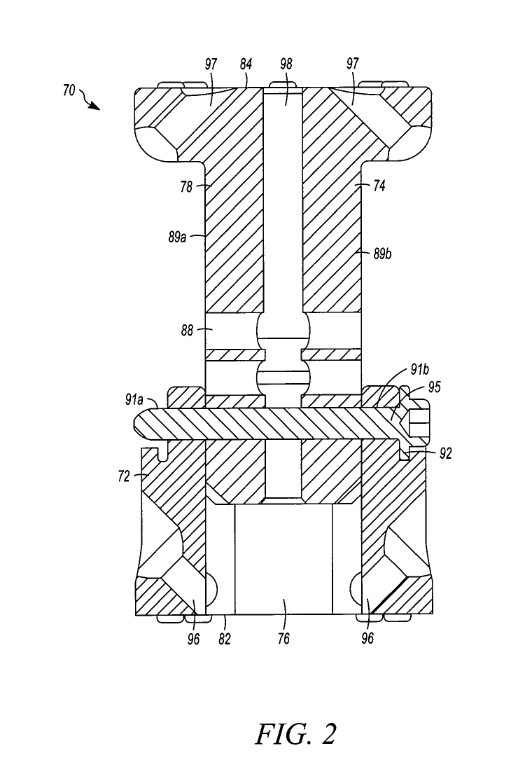

Referring to FIG. 2, the inferior member 72 is provided with a pair of holes 91a extending laterally through one side of the member into the bore 76. A corresponding aligned pair of holes 91b is provided in the opposite side of the inferior member 72. The superior member 74 can be positioned such that any one of the matrix of holes 88 is aligned with corresponding holes 91a, 91b. A pin or staple 95 is provided for insertion into one of the holes 91a, the arms of the pin or staple 95 extending through the wall of the inferior member and into the aligned one of the holes 88. The pin or staple 95 thereby engages both the inferior and superior members 72, 74 in the holes and fixes their relative position. It will be appreciated that the provision of holes or groves in the superior and/or inferior members 72, 74 is convenient for fixating the implant at a required height. However, this may be achieved by other means—for example, a separate insert may be provided that engages with one or other of the inferior or superior members, the insert being provided with the fixating means.

A further feature of this matrix of holes 88 is that it creates a coarse porous structure for potential bony ingrowth.

A clamping and locking mechanism 92 is provided to secure the pin or staple 95 in position and ensure that it cannot subsequently back out and fall out. This clamping and locking mechanism may be a flange on the head of the pin or staple engaging in a groove in the inferior member 72. Alternatively the clamping mechanism may take the form of a screw thread or knurl on the shank of the pin.

The inferior member 72 is provided with openings 93 in the outer wall extending laterally into the body close to the end surface 82. These openings may extend completely through the inferior member 72 or alternatively they may extend into a blind hole. Similar openings 94 are provided close to the end surface 84 of the superior member 74. These openings 94 are used to receive fingers of a distracting instrument (not shown), which will be described in more detail below.

In order to locate the prosthesis into an inter-vertebral space, the fingers of the distracting instrument are inserted into the openings 93, 94 while the prosthesis 70 is in its retracted position, in which the longitudinal portion 78 of the superior member is seated within the bore 76 of the inferior member 72. In this retracted condition the prosthesis 70 is quite short and can easily be inserted into the vertebral space available. The distracting tool is then used to expand the prosthesis 70 in a telescopic fashion. The end surface 82 of the inferior member 72 engages an opposing face of a first (e.g. a lower) vertebral body. As the prosthesis 70 is expanded, the end surface 84 of the superior member 74 will come into contact with the opposing face of a second (e.g. upper) vertebral body. Further expansion of the prosthesis 70 will push the first and second vertebral bodies apart to restore the spine to its normal and/or stable condition and balance. As this occurs, the force required to push the vertebral body apart increases and the surgeon can feel the amount of resistance on the distracting tool and use this to judge when an appropriate amount of expansion has taken place. Moreover, as the force increases, the teeth, grooves or pyramids 85, 86 on the end surfaces 82, 84 grip the bone of the vertebral body to form secure interfaces between the vertebral body and the prosthesis 70.

Once the prosthesis 70 has been expanded to the required height, the pin or staple 95 is inserted to fixate the inferior member 72 in relation to the superior member 74 using indicating features marked or machined onto the implant. A small amount of adjustment may be required so that one of each of the holes 88 in the superior member is in alignment with the holes 91a in the inferior member. Once inserted, the pin or staple 95 is secured by the clamping arrangement 92 to prevent back out.

One or more angled holes 96 extend through the inferior member 72 from the sides, emerging at the end surface 82. These are used for fasteners (e.g. screws) to secure the implant to the inferior vertebral body. Similar angled holes 97 are provided in the superior member 74 to secure it to the superior vertebral body. One or more fastening devices secure the inferior member to the adjoining vertebral body; and one or more fastening devices secure the superior member to the adjoining vertebral body.

As shown in FIG. 2, a longitudinal hole 98 extends through the superior member 74. This is to allow for in-growth of bone material. It may be desirable to insert bone material into the hole 98, and into the lower part of the bore 76 prior to insertion of the prosthesis.

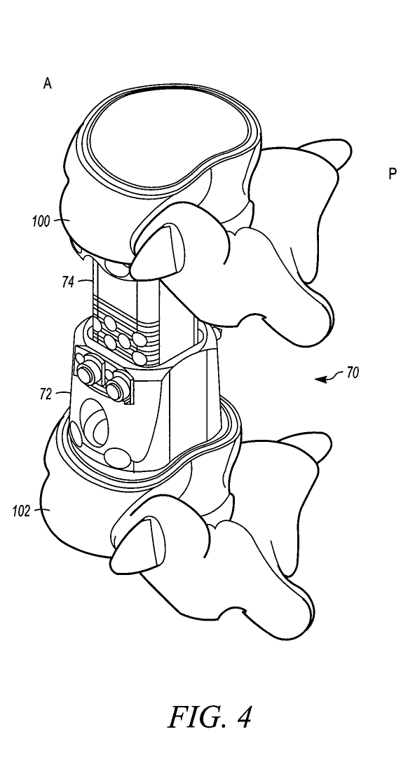

FIG. 4 shows the prosthesis 70 in position between a superior vertebral body 100, and an inferior vertebral body 102. As can be seen, the anterior-posterior (A-P) dimension of the prosthesis is small relative to the A-P dimension of the vertebral body and ensures that the implant sits within the confines of the vertebral bodies to which it is attached. The footprint of the prosthesis may have anterior and posterior faces that are concave. This concavity further reduces the relatively small A-P dimension of the device and allows the Clinician to place graft material alongside on either the anterior or posterior side of the prosthesis.

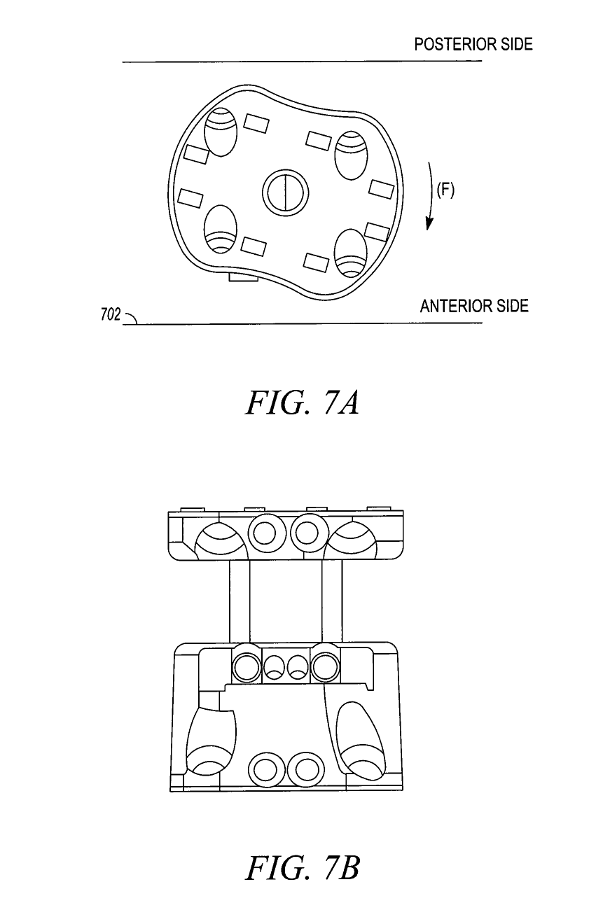

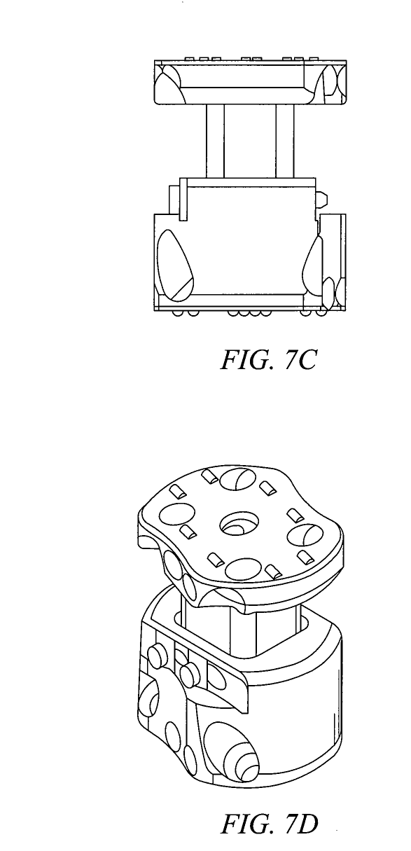

FIGS. 1-6 show an implant that would be implanted from a lateral or oblique surgical approach. An implant may also be provided to have equivalent features but configured in such a way to allow an anterior midline surgical approach. FIG. 7, pertains to an embodiment especially adapted for an anterior midline surgical approach, which is discussed further below.

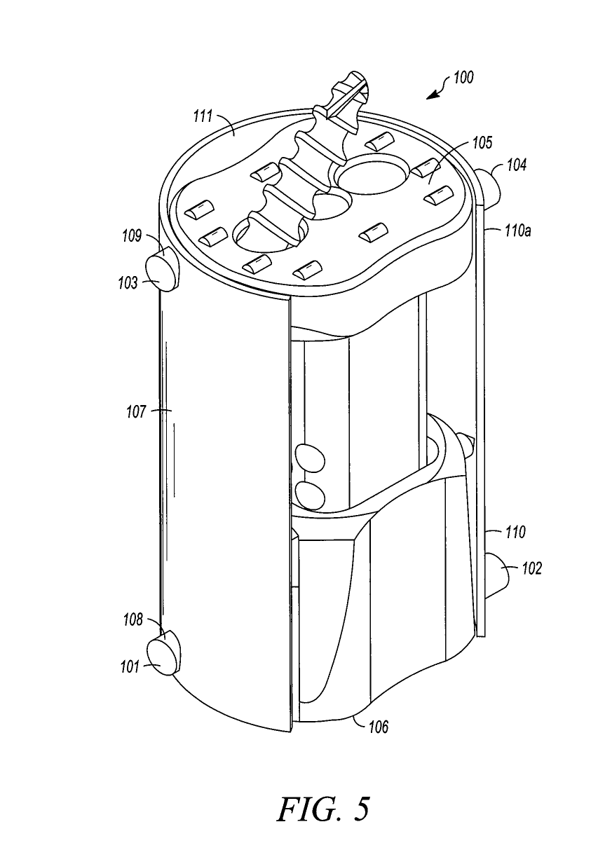

Referring to FIG. 5, a vertebral prosthesis 100, similar to the vertebral prosthesis 70 of FIGS. 1-4, is provided with lugs 101, 102 on each side of the prosthesis 100 close to a lower end face 106. Further lugs 103, 104 are provided on each side close to an upper end face 105. A curtain 107 of braid material has eyelets 108, 109 that allow the curtain to be secured to the lugs 101, 103 on one side of the prosthesis 100. The curtain is provided with further eyelets 110, 110a. The material of the curtain is selected to be compatible with body tissue so as to reduce the likelihood of rejection or infection inside the body (e.g. polyester, PEEK fibres).

Following insertion of the prosthesis into the spine, it is desirable to place bone graft, e.g. autograft, or allograft or synthetic bone, on the anterior face of the prosthesis 100. Bone graft may be of granular form or in shaped rods, for example from the patient's ribs, and positioned alongside the prosthesis 100. These sections of bone will, in time become fused with the vertebral bodies above and below, and will add further strength to the spine.

As can be seen in FIG. 5, the curtain 107 can be used to form an enclosure 111 in the inter-vertebral space surrounding the prosthesis 100 by securing the further eyelets 110, 110a to the lugs 102, 104 on the other side of the prosthesis 100. The enclosure 27 can then be filled with bone graft material, and the prosthesis 100 and curtain 107 forming the enclosure 111 prevent bone graft from falling out. In this way the bone graft will promote bone growth and in time will fuse to the vertebral body, thereby increasing the strength of the spine.

Conveniently, where the prosthesis is expandable (in the manner described above for the prosthesis 100 of FIGS. 1-6), the curtain 107 can be secured to the lugs when the prosthesis is in a retracted condition so that when the prosthesis is expanded the curtain is stretched in the longitudinal direction. This helps to ensure that the enclosure 111 retains its shape in the inter-vertebral space.

A further advantage of the curtain 107 is that it can readily be peeled back by the practitioner if the need should arise—for example if it is desired to adjust the prosthesis or to insert more bone graft material at a later date. The curtain can then be re-attached after such a procedure.

In one embodiment, the curtain 107 is manufactured to a standard size and in such a way that it may be sized intra-operatively e.g. cut with a diatherm. Alternatively the curtain may be made to one size and have the ability to expand. In another alternative, the curtain may contain an elastomeric section to facilitate attachment to the implant and to provide tensioning of the curtain or allowing expansion.

Referring to FIGS. 6a-c, a prosthesis 118 similar to the type shown in FIGS. 1-3 has a series of parallel semi-cylindrical grooves 90 formed transversely across flat sides of a superior member of the prosthesis 118, while a single groove (not visible) is formed across opposing internal surfaces in the bore of the inferior member. The single groove extends into a hole through the wall of the inferior member. As the superior member is raised or lowered relative to the inferior member, the single grooves will become aligned with one of the series of parallel grooves 90, so as to form a cylindrical opening into which a pin or staple can be inserted. As shown in FIG. 6, pins 119 have been inserted.

The prosthesis 118 may be coupled to an anterior rod system 120 by means of an anchoring arrangement 122. The anchoring arrangement 122 is a poly-axial screw coupling that includes a plug member 124, which is received in an opening 126 in the outer surface of an inferior member 121 of the prosthesis 118. The plug member 126 is fixed into the inferior member 121, for example by means of a screw thread. The plug member 124 is coupled to a clamping member 128 by means of an internal screw (not shown), such that the orientation of the plug member 126 relative to the clamping member 128 is variable over a range. The screw can be tightened to fasten the coupling at any orientation within the range. The clamping member 128 is coupled to a longitudinal rod 125 forming part of the anterior rod system 120, and clamped to the longitudinal rod 125 by means of a nut 129, which engages a threaded portion of the clamping member 128. The variable orientation of the poly-axial coupling allows the prosthesis to be anchored to the rod system without requiring precise alignment.

For clarity, FIG. 6c shows only one longitudinal rod 125, whereas more typically the anterior rod system 120 may also use a pair of parallel rods, each coupled to the prosthesis by means of an anchoring arrangement.

The anterior rod system 120 is fixed by means of screws to vertebral bodies above and below the prosthesis 118. In this way the complete assembly of the two vertebral bodies and the prosthesis between them forms a single rigid assembly ensuring that there can be no movement between the prosthesis and the vertebral bodies.

Revision of the prosthesis 118 may be undertaken by reversing the steps above.

Turning to FIG. 7, an embodiment is shown which is especially adapted for implantation via an anterior midline surgical approach. As shown if FIG. 7b, the holes and openings are provided on the embodiment on the anterior side, which facilitates manipulation, extension, and securement of the implant when being accessed from an anterior approach. As seen the top surface is offset (f) slightly which allows the embodiment to be slightly tilted away from the actual midline, which serves to assist in avoiding vital vasculature typically present at the midline. This slight offset is not required and in alternative embodiments, the top surface could be aligned even with the anterior side.

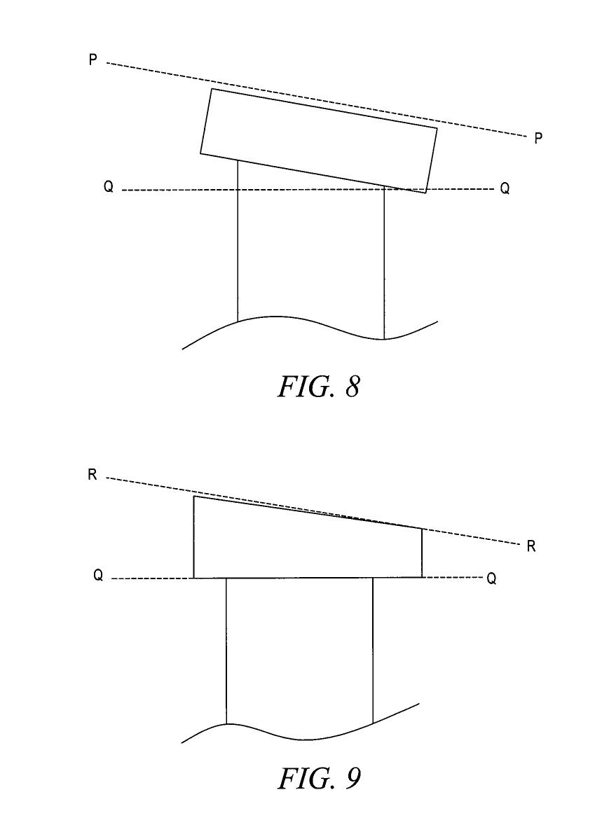

FIG. 8 shows a cross section of a superior member that is configured to provide an angulated superior face. As shown, the plane of the face PP is not parallel to an axial plane QQ of the spine, but rather is angled to facilitate a lordotic or kyphotic angle, depending, on the orientation. FIG. 9 shows an alternative version where the end of the superior member is tapered to form angle RR which is also angulated respective to axial plane QQ. The angulation shown in either FIG. 8 or 9 could be equally implemented on the inferior member surface except turned over to match the angle of the superior end. Furthermore, the angulation of FIG. 8 or 9 may be implemented in any of the embodiments described herein. Typically, whether a lateral version of the implant is used such as shown in FIG. 1-3 or an anterior version such as FIG. 7, the angle will occur from an anterior to posterior, or posterior to anterior direction, which ever is appropriate to conform to the natural curvature of the spine.

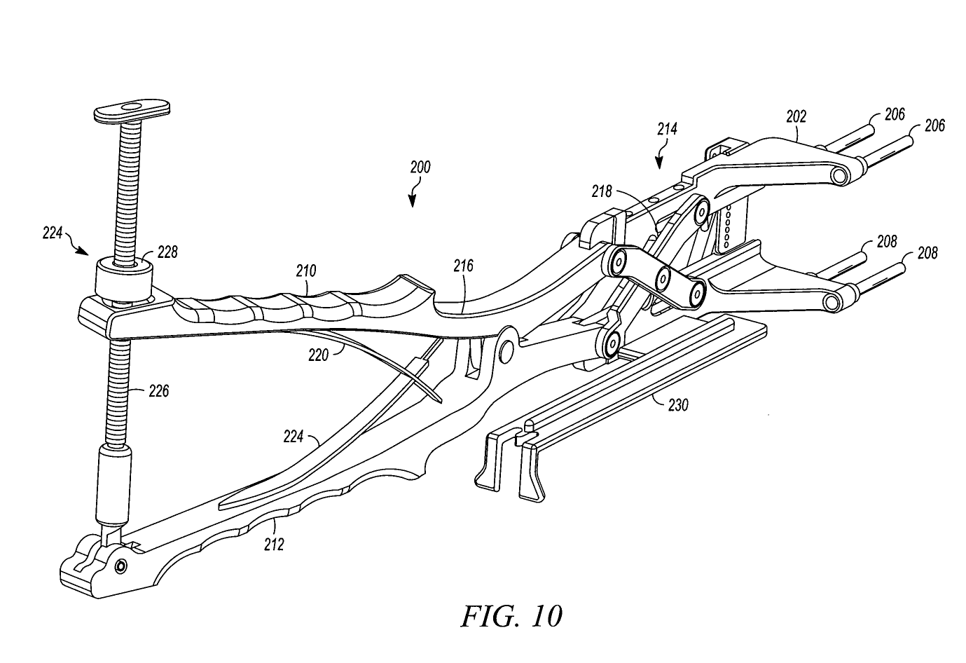

FIG. 10 shows a distracting instrument 200. The instrument 200 includes a superior engagement arm 202 and an inferior engagement arm 204. Each arm 202, 204 has fingers 206, 208 for insertion into corresponding holes in respective superior and inferior members, such as the inferior member 72 and superior member 74 of the prosthesis 70 shown in FIGS. 7 to 9.

The distracting instrument also includes an upper handle 210 and a lower handle 212, which extend distally from the engagement arms 202, 204. A mechanism 214 is provided for moving the engagement arms in a parallel linear movement when upper handle 210 is moved towards the lower handle 212. The mechanism 214 includes a pivot 216 where the upper and lower handles 210, 212 are coupled, and pivoted cross-members 218, which link the engagement arms 202, 204 to respective portions of the handles 210, 212 that extend towards the engagement arms from the pivot 216.

Leaf springs 220, 222 are interposed between the handles 210, 212. A locking mechanism 224 includes a threaded bar 226, which is pivotally attached to the lower handle 212 and extends through a hole in the upper handle 210, and a nut 228. A support member 230 is attached to the instrument alongside the mechanism 214, and is aligned with the fingers 206, 208 of the engagement arms 202, 204.

In use, the practitioner inserts the fingers 206, 208 into respective engagement holes in the superior and inferior members of an expandable implant (for example into the holes 93, 94 of the prosthesis 70 of FIGS. 7 to 9). The implant is preferably in a fully retracted position and is inserted into the space between a pair of vertebral bodies. The practitioner then squeezes the upper and lower handles 210, 212 together such that the mechanism 214 causes the engagement arms 202, 204 to move apart. This expands the implant and distracts the space between the vertebral bodies. The practitioner can feel the resistance to the distraction by the force needed to squeeze the handles together. The leaf springs 220, 222, will add a little to the force, but the main function of these is to ensure that when the practitioner releases the squeezing pressure, the handles move apart again to retract the implant.

Selection of the implant may achieved by use of a sizing gauge (not shown) integral with the locking mechanism 224.

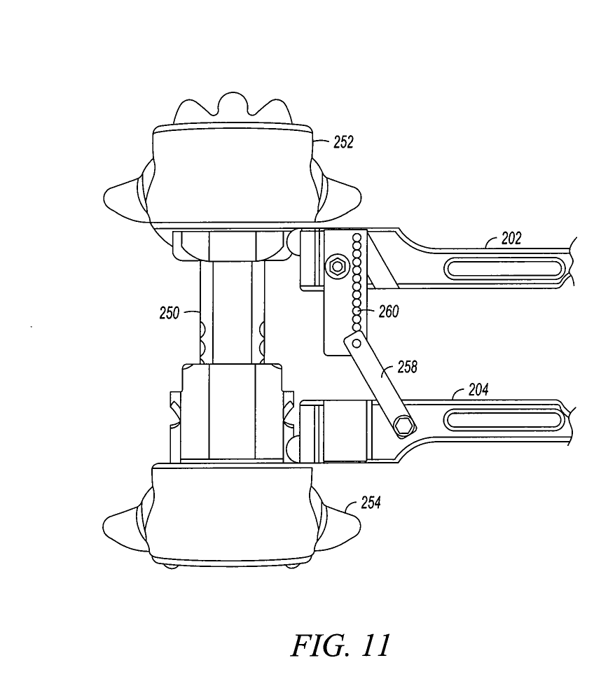

FIG. 11 illustrates the expansion of an implant 250 between upper and lower vertebral bodies 252, 254 by the engagement arms 202, 204. An adjustable limiting device includes a pivoted link member 258 attached to the lower arm 202 and a chain link 260 attached to the upper arm 204 ensures that the instrument cannot inadvertently be used to over-distract the space between the vertebral bodies 232, 234.

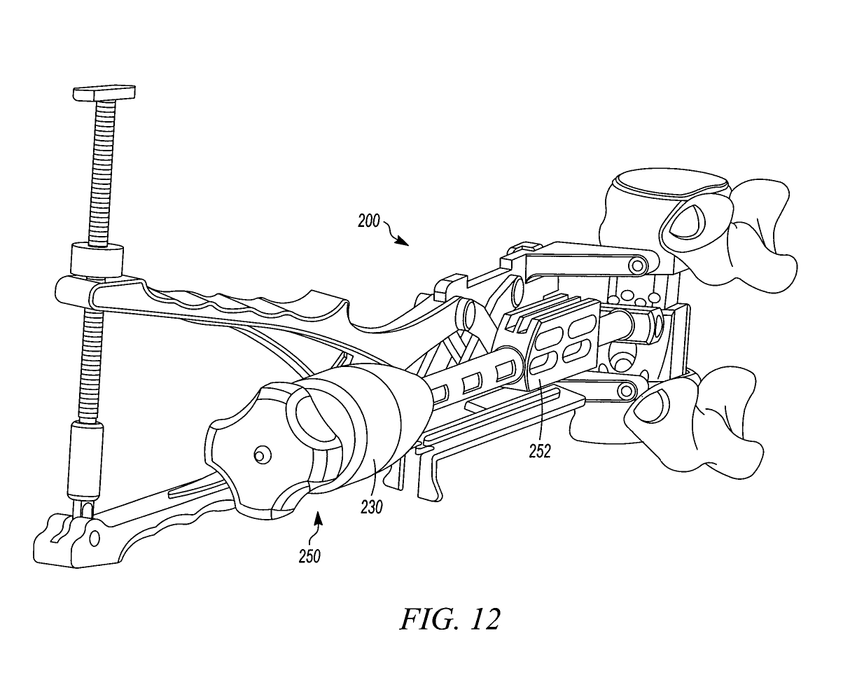

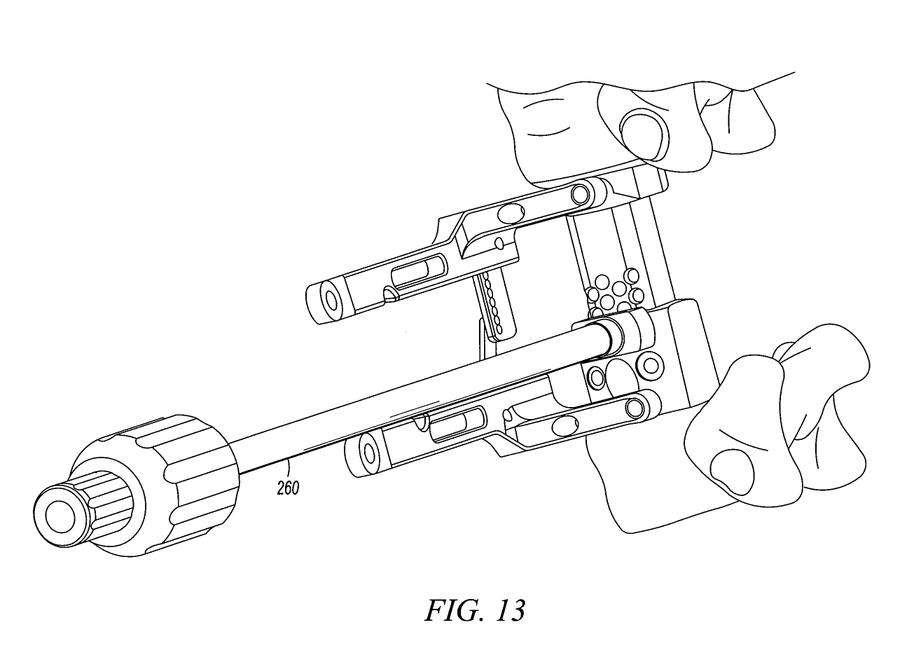

The practitioner can judge a correct or desired amount of distraction by feeling the force needed to squeeze the handles together. This sensory feedback relates to one of the advantageous features of the instrument embodiment. The instrument is then locked in that position by screwing the nut 228 down so that it contacts the top of the upper handle 210. The practitioner can now fix the implant by insertion of fasteners, as depicted in FIG. 12. A fastening tool 250 includes a gripping device 252, which holds the fastener (screw, pin, staple, bollard etc.). The fastening tool 250 is supported on the support member 230, which also guides the insertion tool so that the fastener is inserted directly into a fastener opening on the implant. FIG. 13 shows a perspective view of another fastening tool device 260 that may be used. Most of the instrument has been disassembled in FIG. 13. More clearly shown in FIG. 13 is how arms 202, 204 extend laterally from the rest of the instrument and are spread apart upon actuation. This provides an access space for the surgeon to access the implant for manipulation, fastening, securement, delivery of biological material, etc.

Once the implant has been fixated in position the instrument can be removed by simply pulling it away from the implant so that the fingers 206, 208 slide out of the holes in the superior and inferior members of the implant. The fingers are shown as being cylindrical but it is contemplated that any suitable geometric configuration including, but not limited to, structures with an elliptical or orthogonal cross-sections can be implemented.

Although the above discussion discloses various exemplary embodiments of the invention, it should be apparent that those skilled in the art can make various modifications that will achieve some of the advantages of the invention without departing from the true scope of the invention. U.S. Pat. Nos. 5,776,198; 6,524,341; 6,176,881 and U.S. Patent Pub. 2004/0199252 are cited for background purposes. The teachings of all references cited herein are incorporated in their entirety to the extent not inconsistent with the teachings herein.

1. An implant comprising:
an inferior member having an inferior end surface for engaging a superior face of an inferior vertebral body and a longitudinal portion;
a superior member having a superior end surface for engaging an opposing inferior surface of a second vertebral body, and a portion adapted to cooperate with the longitudinal portion of the inferior member such that the superior member is moveable relative to the inferior member by sliding in the longitudinal direction; and
fixating means for securing the superior member to the inferior member.

2. The implant of claim 1 wherein either the longitudinal portion of the inferior member or the cooperating portion of the superior member is an open-ended portion having a bore that receives the cooperating portion of the other member, so as to provide a close sliding fit.

3. The implant of claim 1 wherein the fixating means between the inferior and superior members is configured to allow adjustment of the height of the implant by securing the superior member to the inferior member at positions between a maximum and a minimum height.

4. The implant of claim 1 wherein each member of the implant is fixable directly to the adjacent superior and inferior vertebral bodies.

5. The implant of any of claims 1, wherein the implant is reversible so as to be capable of placement in either an anterior orientation or a posterior orientation

6. The implant of claim 6 wherein, in the anterior orientation the implant provides a lordotic angle between the superior and inferior vertebral bodies, and, when reversed into the posterior orientation, provides a kyphotic angle and upon implantation via an anterior midline approach the implant is geometrically configured to promote angulation corresponding to the natural curvature of the spine.

7. The implant of claim 6, wherein the inferior member surface and/or the superior member surface is angled relative to an axial plane of the spine.

8. The implant of claim 1, wherein the superior and inferior members have a symmetry of shape and angle of their respective end surfaces such that the implant provides a blend of kyphotic and lordotic angles.

9. The implant of claim 8 configured to provide a lordotic angle on the superior end surface and a kyphotic angle on the inferior end surface.

10. The implant of any of claim 1, wherein the superior and inferior members provide a normal or neutral angle.

11. The implant of claim 1, wherein the first and second members are formed of a radio-translucent material, such as polyether-etherketone (PEEK).

12. The implant of claim 1, wherein the first and second members are formed of a polymer, polymer composite, bio-resorbable or other biomaterials.

13. The implant of claim 12, wherein the material exhibits the characteristics of Young's Modulus close to that of bone.

14. The implant of claim 13, wherein the material is a synthetic bone substitute, bioglass or ceramic material that is resorbable and/or osteoinductive and/or osteostimulative and/or osteoconductive.

15. The implant of claim 1, wherein the cross-sections of the inferior and superior members are shaped to resist rotation relative to each other and relative to the adjacent bone structures.

16. The implant of claim 1, wherein the fixating means comprises a clip or spring or bolt, threaded or unthreaded, or pin(s) or staple(s) or other fasteners of similar shape and function.

17. The implant of claim 16 configured for insertion of the fixating means into a hole or holes in the wall of the longitudinal portion of the inferior member so as to engage into a hole or holes formed in the longitudinal portion of the superior member.

18. The implant of claim 16 comprising partial holes, grooves or channels formed in the cooperating surfaces of the longitudinal portions of the inferior and superior members for receiving the fixating means.

19. The implant of claim 18 wherein the cross-section of the longitudinal portion of the superior member has a pair of substantially parallel flat faces, each face including a plurality of preformed grooves extending laterally across the face, the preformed grooves being sized to receive an arm of the bolt or pin or staple.

20. The implant of claim 19 wherein the holes or grooves or channel are reinforced with a composite material or sleeve or similar.

21. The implant of claim 18 wherein the superior and/or inferior members are provided with an array of holes or grooves or channels to provide a range of insertion locations defined by alignment of one or more holes/grooves in the superior member with one or more holes/grooves in the inferior member.

22. The implant of claim 21 wherein the holes or grooves or channels and pin or staple may be of any suitable cross section.

23. The implant of claim 22, wherein the pin comprises a flanged head that mates with a small groove defined in the inferior member, wherein mating of flanged head with the small groove facilitates retention of the pin.

24. The implant of claim 21, wherein the holes or grooves or channels are spaced at intervals of up to approximately 3.5 mm, allowing adjustment of the height in increments of up to approximately 3.5 mm.

25. The implant of claim 21, wherein the holes or grooves or channels are spaced at intervals of between about 2.25 to about 2.75 mm.

26. The implant of 1, wherein the fixating means is lockable in position with a primary or secondary locking device.

27. The implant of claim 1, wherein anchoring means are provided for anchoring the implant to an anterior or lateral plate or rod system, and wherein the anchoring means comprises an opening in the first and/or the second member for receiving a coupling assembly.

28. The implant of claim 27, wherein the coupling assembly is a poly-axial coupling assembly.

29. The implant of claim 27 wherein the poly-axial coupling assembly includes a clamping arrangement for securing the coupling to a longitudinal rod or bar forming part of the anterior plate or rod system and/or wherein the anchoring means comprises a hinged joint between the members and plate.

30. The implant of claim 27, wherein the coupling assembly is a fixed head coupling assembly.

31. The implant of claim 1, further comprising openings provided in the respective member for an expansion tool to engage the first and second members so as to move the members to increase or decrease the height of the implant prior to fixation.

32. A method of repairing a damaged body structure between a superior vertebral body and an inferior vertebral body, the method comprising:
preparing an insertion site adjacent the superior and inferior vertebral bodies;
inserting the implant of claim 1 adjacent the superior and inferior vertebral bodies; and
securing the inferior member to the inferior vertebral body and the superior member to the superior vertebral body.

33-53. (canceled)
